(19) United States
(12) Patent Application Publication
(10) Pub. No.: US 2001/0046990 A1
(43) Pub. Date: Nov. 29, 2001

(54) Pharmaceutical composition and the process for its preparation

(76) Inventors: Thyge Borup Hjorth, Farum (DK); Breian Knudsen, Albertslund (DK)

Correspondence Address: Nova Nordisk of North America, Inc., 405 Lexington Avenue, Suite 6400, New York, NY, 10174-6401, US

(21) Appl. No.: 09/826,245

(22) Filed: Apr. 4, 2001

Related U.S. Application Data
(60) Provisional application No. 60/196,981, filed on Apr. 13, 2000.

(30) Foreign Application Priority Data
Apr. 4, 2000 (DK) PA 2000 00557

(51) Int. Cl. A61K 31/538

(52) U.S. Cl. 514/229.8

(57) ABSTRACT

A new stable pharmaceutical composition containing (−) 3-[4-[2-Phenoxazin-10-yl)ethoxy]phenyl]-2-ethoxypropanoic acid as active ingredient is provided, as well as process methods and formulations.

### CROSS-REFERENCE TO RELATED APPLICATION

This application claims priority under 35 U.S.C. of 119 of United States provisional application No. 60/196,981 filed on Apr. 13, 2001 and Danish application no. PA 2000 00557 filed on Apr. 4, 2001, the contents of which are fully incorporated herein by reference.

### FIELD OF THE INVENTION

The subject-matter of the present invention is a new pharmaceutical composition containing (−) 3-[4-[2-phenoxazin-10-yl)ethoxy]phenyl]-2-ethoxypropanoic acid as active ingredient or a pharmaceutically acceptable salt thereof and the process for its preparation.

### BACKGROUND OF THE INVENTION

(−) 3-[4-[2-Phenoxazin-10-yl)ethoxy]phenyl]-2-ethoxypropanoic acid and pharmaceutically acceptable salts thereof has been found useful in the treatment of type 2 diabetes acting as an insulin sensitizer as disclosed in PCT Publication WO 99/19313.

In WO 99/19313 the active ingredient is present as the base or as a pharmaceutically acceptable salt, preferably as the sodium salt.

### SUMMARY OF THE INVENTION

The aim of the present invention is to provide a new compositions intended for the preparation of (−) 3-[4-[2-phenoxazin-10-yl)ethoxy]phenyl]-2-ethoxypropanoic acid or of one of its pharmaceutically acceptable salts with improved stability, in particular solid dosage forms thereof.

### DESCRIPTION OF THE INVENTION

It has been found in fact that (−) 3-[4-[2-phenoxazin-10-yl)ethoxy]phenyl]-2-ethoxypropanoic acid and its pharmaceutically acceptable salts may decompose in the presence of and in contact with water. Further it has been observed that decomposing may occur in the presence of oxygen.

Thus, from a first aspect, the subject-matter of the present invention is a pharmaceutical composition intended for the preparation of dosage forms and in particular solid dosage forms containing an efficacious quantity of (−) 3-[4-[2-phenoxazin-10-yl)ethoxy]phenyl]-2-ethoxypropanoic acid or of one of its pharmaceutically acceptable salts as active ingredient.

The present invention is based on the surprising discovery of the fact that the stability of (−) 3-[4-[2-phenoxazin-10-yl)ethoxy]phenyl]-2-ethoxypropanoic acid, or of one of its pharmaceutically acceptable salts, can be considerably improved in preparations containing (−) 3-[4-[2-phenoxazin-10-yl)ethoxy]phenyl]-2-ethoxypropanoic acid or of its pharmaceutically acceptable salts and antioxidant agent if the product is composed of excipients which do not contain water.

Another characteristic of the invention is, that a surprisingly very high degree of mixing homogeneity can be obtained with (−) 3-[4-[2-phenoxazin-10-yl)ethoxy]phenyl]-2-ethoxypropanoic acid or a pharmaceutically acceptable salt thereof in low concentrations in powder and tablet formulations using a certain combination of pharmaceutical fillers, adjuvants and mixing process.

Pharmaceutically acceptable salts forming part of this invention include salts such as alkali metal salts like Li, Na, and K salts, alkaline earth metal salts like Ca and Mg salts, salts of organic bases such as lysine, arginine, guanidine, diethanolamine, choline and the like, ammonium or substituted ammonium salts, aluminium salts. Salts may include acid addition salts where appropriate which are, sulphates, nitrates, phosphates, perchlorates, borates, hydrohalides, acetates, tartrates, maleates, citrates, succinates, palmoates, methanesulplionates, benzoates, salicylates, hydroxynaphthoates, benzenesulfonates, ascorbates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. In a preferred embodiment, (−) 3-[4-[2-phenoxazin-10-yl)ethoxy]phenyl]-2-ethoxypropanoic acid, arginine is used in the present invention.

(−) 3-[4-[2-Phenoxazin-10-yl)ethoxy]phenyl]-2-ethoxypropanoic acid, together with a conventional adjuvant, antioxidant carrier, or diluent, and if desired a pharmaceutically acceptable acid addition salt thereof, may be placed into the form of pharmaceutical compositions and unit dosages thereof, and in such form may be employed as solids, such as tablets or filled capsules, or oral powders to be diluted immediately before use filled with the same, all for oral use, in the form of suppositories for rectal administration; or as pessaries for vaginal use; or in the form of sterile injectable powders for parenteral, transdermal, nasal, pulmonary and ocular use.

Within the framework of the present description and of the claims, by powders is meant any mixture of components, granulated or not, intended to be placed in solution and/or in suspension in water, or again to be ingested directly or by any other appropriate means as for example in a mixture with a food product.

In accordance with a particular characteristic of the invention, the manufacture of tablets are carried out as a direct compression.

In accordance with another particular characteristic, this composition also contains pharmaceutically acceptable excipients.

In accordance with a particular characteristic of the invention, the antioxidant agent cited above is selected from among α-tocopherol, γ-tocopherol, δ-tocopherol, extracts of natural origin rich in tocopherol, L-ascorbic acid and its sodium or calcium salts, ascorbyl palmitate, propyl gallate (PG), octyl gallate, dodecyl gallate, butylated hydroxy anisole (BHA) and butylated hydroxy toluene (BHT).

In accordance with a currently preferred embodiment, the antioxidant agent will be α-tocopherol.

In accordance with another particular characteristic of the invention, the diluent is lactose and/or cellulose microcrystalline, magnesium stearate, talc.

However, any other pharmaceutically acceptable diluents could be used if the diluents has a low water content.

The quantities of diluents can be easily determined by a person skilled in the art and depend of course on the final pharmaceutical form required. Generally speaking, a composition which complies with the present invention and which are intended for the preparation of tablets, may contain, expressed in parts by weight per 100 parts of (−) 3-[4-[2-phenoxazin-10-yl)ethoxy]phenyl]-2-ethoxypropanoic acid, or of one of its pharmaceutically acceptable salts:

between 100 and 400,000 parts by weight of anhydrous lactose;

between 100 and 400,000 parts by weight of lactose monohydrate

between 100 and 400,000 parts by weight of dibasic calciiumphosphate

between 1 and 100 parts by weight of an antioxidant;

between 50 and 500 parts by weight of pregelatinized starch;

between 1000 and 10,000 parts by weight of microcrystalline cellulose;

between 10 and 500 parts by weight of crospovidone;

between 10 and 500 parts by weight of silicon dioxide;

between 10 and 500 parts by weight of hydrogenated vegetable oil;

between 10 and 500 parts by weight of magnesium stearate;

between 10 and 500 parts by weight of hydroxypropyl methylcellulose;

between 10 and 500 parts by weight of hydroxypropyl cellulose;

between 1000 and 10,000 parts by weight of mannitol;

between 10 and 500 parts by weight of stearic acid;

between 10 and 500 parts by weight of titanium dioxide;

According to a preferred embodiment of the invention the water content of the excipients is very low. More specifically the water content in the diluents is very low in order to minimize the water content of the pharmaceutical composition. Lactose is used in its anhydrous form.

Herein a vary low water content or a low water content is a content of water below about 1%, preferably below about 0.5%, and even more preferably below about 0.1% (weight/weight).

Furthermore, all excipients may be applied in a dry form.

In accordance with a second aspect, the subject-matter of the present invention is a pharmaceutical preparation, in the form of tablet or powder, characterised in that it contains a composition as defined previously associated if required with at least one customary additive selected from among the sweeteners, flavouring agents, colours and lubricants.

Another manufacturing process for pharmaceutical compositions according to the invention is mixing of (−) 3-[4-[2-phenoxazin-10-yl)ethoxy]phenyl]-2-ethoxypropanoic acid or a pharmaceutically acceptable salt thereof, one or more antioxidants and other pharmaceutical excipients followed by melt granulation in a high shear mixer. Hydrogenated, vegetable oil, waxes or other low temperature melting binders can be used. The granules can be filled into capsules, compressed into tablets or used in other pharmaceutical dosage forms.

More preferably the manufacturing process applied is direct compression of tablets, wherein (−) 3-[4-[2-phenoxazin-10-yl)ethoxy]phenyl]-2-ethoxypropanoic acid or a pharmaceutically acceptable salt thereof, one or more antioxidants and other excipients suitable for direct compression are mixed followed by tabletting.

Yet another preferred embodiment of the manufacturing process is wet granulation, where granules are obtained by wet massing of (−) 3-[4-[2-phenoxazin-10-yl)ethoxy]phenyl]2-ethoxypropanoic acid or a pharmaceutically acceptable salt thereof, together with one or more antioxidants and other excipients.

It is assumed that the contact time with water has to be very short.

The most preferred process comprises the direct compression whereby (−) 3-[4-[2phenoxazin-10-yl)ethoxy]phenyl]-2-ethoxypropanoic acid or a pharmaceutically acceptable salt thereof is kept at conditions of low water vapour pressure.

Herein the term low water vapour pressure designates a relative humidity below about 40 %, preferably below about 30%. In practice, it may be difficult to obtain a relative humidity below about 10%.

A low oxygen pressure can be obtained by using argon or nitrogen. Herein a low oxygen pressure also is presenting an atmosphere containing below about 10% oxygen, preferably below about 5% oxygen, even more preferred below about 1% oxygen (volume/volume).

A sweetener may be a natural sugar such as sorbitol or a synthetic product such as saccharine or aspartame.

When the antioxidant selected is ascorbylpalmitat, propylgallat, which is a powder, it can be advantageous to mix it in an appropriate excipient such as α-tocopherol succinat, lactose or cellulose micrycrystalline.

The present invention will further be illustrated with the following non-exhaustive examples.

### EXAMPLES

### EXAMPLE 1: (−) 3-[4-[2-Phenoxazin-10-yl)ethoxy]phenyl]-2-ethoxypropanoic acid 0.5 mg

(−) 3-[4-[2-Phenoxazin-10-yl)ethoxy]phenyl]-2-ethoxypropanoic acid ,arginine 0.353%

Cellulose Microcrystallline 20%

Lactose 75%

Magnesium Stearate 0.5%

Talc 4.5%

The tablets were prepared by the following procedure: The active ingredient is mixed with cellulose microcrystalline by hand. Lactose is added and the mixing continues in a drum mixer for 5 minutes. The talc is added and the mixing continues for 2 minutes. The magnesium stearate is added and the mixing continues for 1 minute more.

### EXAMPLE 2: (−) 3-[4-[2-Phenoxazin-10-yl)ethoxy]phenyl]-2-ethoxypropanoic acid 10 mg (−) 3-[4-[2-Phenoxazin-10-yl)ethoxy]phenyl]-2-ethoxypropanoic acid,arginine 7.075%

Cellulose Microcrystalline 20%

Lactose 67.95%

Magnesium Stearate 0.5%

Talc 4.5%

### EXAMPLE 3: (−) 3-[4-[2-Phenoxazin-10-yl)ethoxy]phenyl-2-ethoxypropanoic acid, 0.1 mg with a total mass of 80 mg

(−) 3-[4-[2-Phenoxazin-10-yl)ethoxy]phenyl-2-ethoxypropanoic acid, arginine 0.18%

Tablettose 80 96.12%

Avicel PH 102 3.00%

Cab-Osil M-3 0.20%

Magnesium Stearate 0.50%

At higher strengths the amount of (−) 3-[4-[2-Phenoxazin-10-yl)ethoxy]phenyl-2-ethoxypropanoic acid arginine will be subtracted from Tablettose 80.

Manufacturing procedure:

The active ingredient is sieved through a 0.125 to 0.4 mm sieve and mixed with the same amount of Tablettose 80. Cab-Osil is sieved through a 1.0 mm sieve together with a small amount of Tablettose. The active ingredient, Tablettose, Avicel and Cab-Osil are mixed in a drum mixer in the range of 20 to 30 minutes depending on the strengths manufactured. Magnesium Stearate is sieved through a 0.125 mm sieve immediately before use and is mixed with the other ingredients in a drum mixer for 3 more minutes.

### EXAMPLE 4: (−) 3-[4-[2-Phenoxazin-10-yl)ethoxy]phenyl-2-ethoxypropanoic acid, 0.5 mg

(−) 3-[4-[2-Phenoxazin-10-yl)ethoxy]phenyl-2-ethoxypropanoic acid, arginine 0.353%

Lactose 87.65%

Polyethylenglycol 6000 7%

Talc 5%

The granulate is manufactured in a Diosna 1 L high-shear mixer—using a water bath of 70° C. The mixing is carried out at 2000 RPM, chopper 1600 RPM and the granulation is performed at approx. 70° C. The hot granulate is sieved through sieve 1.00 mm, and the cold granulate through sieve 1000 mm. The glidant is added with a card for 2 min. The tablets are manufactured using a Korsch tabletmachine with ovale punch.

### EXAMPLE 5: (−) 3-[4-[2-Phenoxazin-10-yl)ethoxy]phenyl-2-ethoxypropanoic acid, 10 mg

(−) 3-[4-[2-Phenoxazin-10-yl)ethoxy]phenyl-2-ethoxypropanoic acid, arginine 7.075%

Lactose 80.95%

Polyethylenglycol 6000 7%

Talc 5%

The granulate is manufactured in a Diosna 1 L high-shear mixer—using a water bath of 70° C. The mixing is carried out at 2000 RPM, chopper 1600 RPM and the granulation is performed at approx. 70° C. The hot granulate is sieved through sieve 1.00 mm, and the cold granulate through sieve 1000 mm. The tablets were prepared as described in Example 1. The glidant is added with a card for 2 min. The tablets are manufactured using a Korsch tabletmachine with ovale punch.

1. Pharmaceutical composition comprising
(−) 3-[4-[2-phenoxazin-10-yl)ethoxy]phenyl]-2-ethoxypropanoic acid or a pharmaceutically acceptable salt thereof, and optionally a pharmaceutically acceptable carrier.

2. The composition of claim 1 in the form of a tablet, a powder or a capsule.

3. A process for the preparation of the composition of claim 1, comprising the step of forming a mixture of (−) 3-[4-[2-phenoxazin-10-yl)ethoxy]phenyl]-2-ethoxypropanoic acid or a pharmaceutically acceptable salt thereof, and one or more pharmaceutically acceptable carriers.

4. The process of claim 3, further comprising the step of compressing the mixture with excipients of a low water content.

5. The process of claim 4, characterized in that the steps are carried out at low water vapour pressure and low oxygen pressure.

6. A pharmaceutical composition comprising
(−) 3-[4-[2-phenoxazin-10-yl)ethoxy]phenyl]-2-ethoxypropanoic acid or a pharmaceutically acceptable salt thereof, and pharmaceutically acceptable excipients with low water content and an antioxidant.

7. The pharmaceutical composition of claim 6 in the form of a tablet, a powder or a capsule.

8. The pharmaceutical composition of claim 6 comprising, expressed in parts by weight per 100 parts of (−) 3-[4-[2-phenoxazin-10-yl)ethoxy]phenyl]-2-ethoxypropanoic acid, or of one of its pharmaceutically acceptable salts, and between 1 and 100 parts by weight of an antioxidant and a pharmaceutically acceptable excipients selected from the group consisting of:
between 100 and 400,000 parts by weight of anhydrous lactose,
between 100 and 400,000 parts by weight of lactose monohydrate
between 100 and 400,000 parts by weight of dibasic calciiumphosphate
between 50 and 500 parts by weight of pregelatinized starch,
between 1000 and 10,000 parts by weight of microcrystalline cellulose,
between 10 and 500 parts by weight of crospovidone,
between 10 and 500 parts by weight of silicon dioxide,
between 10 and 500 parts by weight of hydrogenated vegetable oil,
between 10 and 500 parts by weight of magnesium stearate,
between 10 and 500 parts by weight of hydroxypropyl methylcellulose,
between 10 and 500 parts by weight of hydroxypropyl cellulose,
between 1000 and 10,000 parts by weight of mannitol,
between 10 and 500 parts by weight of stearic acid, and
between 10 and 500 parts by weight of titanium dioxide.

9. The pharmaceutical composition of claim 6, wherein the pharmaceutically acceptable excipients are selected from the group consisting of:
between 100 and 400,000 parts by weight of anhydrous lactose,
between 100 and 400,000 parts by weight of lactose monohydrate
between 100 and 400,000 parts by weight of dibasic calciiumphosphate
between 50 and 500 parts by weight of pregelatinized starch,
between 1000 and 10,000 parts by weight of microcrystalline cellulose,
between 10 and 500 parts by weight of crospovidone,
between 10 and 500 parts by weight of silicon dioxide,
between 10 and 500 parts by weight of hydrogenated vegetable oil,
between 10 and 500 parts by weight of magnesium stearate,
between 10 and 500 parts by weight of hydroxypropyl methylcellulose,
between 10 and 500 parts by weight of hydroxypropyl cellulose,
between 1000 and 10,000 parts by weight of mannitol,
between 10 and 500 parts by weight of stearic acid, and
between 10 and 500 parts by weight of titanium dioxide,
expressed in parts by weight per 100 parts of (−) 3-[4-[2-phenoxazin-10-yl)ethoxy]phenyl]-2-ethoxypropanoic acid, or of one of its pharmaceutically acceptable salts.

10. The pharmaceutical composition of claim 6, wherein the pharmaceutically acceptable excipients are selected from the from the group consisting of:
lactose and/or cellulose microcrystalline, magnesium stearate, and talc.

11. The pharmaceutical composition of claim 6, wherein the pharmaceutically acceptable excipients have a low water content.

12. The pharmaceutical composition of claim 6, wherein the pharmaceutically acceptable excipients have a very low water content.

13. The pharmaceutical composition of claim 6, wherein the pharmaceutically acceptable excipients are in a dry form.

14. The pharmaceutical composition of claim 6, wherein the antioxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate (PG), octyl gallate, dodecyl gallate, butylated hydroxy anisole (BHA) or butylated hydroxy toluene (BHT).

15. The pharmaceutical composition of claim 14, wherein the antioxidant is α-tocopherol.

16. The pharmaceutical composition of claim 6, associated with at least one customary additive selected from among the sweeteners, flavouring agents, colours and lubricants.

17. A process for the preparation of a composition of claim 6 comprising the step of (a) forming a mixture of (−) 3-[4-[2-phenoxazin-10-yl)ethoxy]phenyl]-2-ethoxypropanoic acid or a pharmaceutically acceptable salt thereof, and one or more pharmaceutically acceptable excipients and an antioxidant.

18. The process of claim 17, further comprising the step of (b) compressing the mixture.

19. The process of claim 17, characterized in that the step is carried out at low water vapour pressure and low oxygen pressure.

20. A pharmaceutical composition comprising:
(−) 3-[4-[2-phenoxazin-10-yl)ethoxy]phenyl]-2-ethoxypropanoic acid, arginine 0.353%
Cellulose Microcrystallline 20%
Lactose 75%
Magnesium Stearate 0.5%
Talc 4.5%

21. A pharmaceutical composition comprising:
(−) 3-[4-[2-phenoxazin-10-yl)ethoxy]phenyl]-2-ethoxypropanoic acid, arginine 7.075%
Cellulose Microcrystalline 20%
Mannitol 6.95%
Magnesium Stearate 0.5%
Talc 4.5%

22. A pharmaceutical composition comprising:
(−) 3-[4-[2-phenoxazin-10-yl)ethoxy]phenyl]-2-ethoxypropanoic acid, arginine 0.18%
Tablettose 80 96.12%
Avicel PH 102 3.00%
Cab-Osil M-3 0.20%
Magnesium Stearate 0.50%

23. A pharmaceutical composition comprising:
(−) 3-[4-[2-phenoxazin-10-yl)ethoxy]phenyl-2-ethoxypropanoic acid, arginine 0.353%
Lactose 87.65%
Polyethylenglycol 6000 7%
Talc 5%

24. A pharmaceutical composition comprising:
(−) 3-[4-[2-phenoxazin-10-yl)ethoxy]phenyl-2-ethoxypropanoic acid, arginine 7.075%
Lactose 80.95%
Polyethylenglycol 6000 7%
Talc 5%

25. The pharmaceutical composition of claim 20, wherein (−) 3-[4-[2-phenoxazin-10-yl)ethoxy]phenyl]-2-ethoxypropanoic acid arginine is used.
